(19) United States
(12) Patent Application Publication
(10) Pub. No.: US 2022/0218559 A1
(43) Pub. Date: Jul. 14, 2022

(54) Grounded Massage Tool

(71) Applicant: Jeremy Madvin, Westlake Village, CA (US)

(72) Inventor: Jeremy Madvin, Westlake Village, CA (US)

(21) Appl. No.: 17/148,566

(22) Filed: Jan. 14, 2021

(51) Int. Cl. A61H 15/00

(52) U.S. Cl. CPC A61H 15/00; A61H 2015/0035; A61H 2201/0153; A61H 2201/10; A61H 2201/1253; A61H 2201/1695

(57) ABSTRACT

The present invention relates to the field of family health care, in particular to a manual massager with grounding benefits. The highly portable and manual device transfers the user's body excess voltage and transfers through the wire to grounding port and thus deposited to Earth. The assembly incorporates the benefits of grounding (earthing) with the benefits of massage therapy. The manual massager has a simple structure, low manufacturing cost and controllable massaging force, and is convenient to use.

### COPYRIGHT NOTICE

A portion of the disclosure of this patent document contains material which is subject to copyright protection. The copyright owner has no objection to the facsimile reproduction by anyone of the patent disclosure, as it appears in the Patent and Trademark Office patent files or records, but otherwise reserves all copyright rights whatsoever.

### BACKGROUND

### Field of the Invention

This invention relates generally to any manual massage tool and, more particularly, it relates to a portable massage tool, which incorporates the benefits of grounding (earthing) along the benefits of massage therapy.

### Description of the Related Art

The massaging of various parts of the body is an old and well-known art. It is well known that massaging relaxes the muscles, improves circulation, and generally improves the feeling of wellbeing of an individual or patient. It is also believed that massaging has major health benefits. In fact, massage therapy is a popular way to treat a variety of ailments including sore or tired muscles, headaches, minor injuries, stress, poor blood circulation, chronic pain syndromes, and decreases injury rehabilitation time.

Perhaps the oldest and most well known technique of massaging is by the use of the hands and fingers of the massage therapist. While hand and finger massaging is believed to be most beneficial, the hand or finger massage is exhausting for the massage therapist and cannot be self applied without additional stress to the hands. Continuous massaging stresses the fingers, the thumbs, the hands, and the wrist. For this reason, certain implements have been developed in an effort to provide somewhat equivalent benefit to hand or finger massage and also to enable self-massaging. Unfortunately, one problem with the conventional massage tools is that they were not comfortable for the massage therapist to hold and manipulate.

There are multiple inventions that have been proposed in prior art regarding advancements in similar field. For instance, a Korean patent 1,016,88914B1 on Massage tool is issued to Korean inventor. The present invention provides a face skin beautifying vibration massage tool capable of performing massage and improving skin to be modified. To this end, the face skin beautifying vibration massage tool according to the present invention comprises: a body having a contact part formed on the bottom surface thereof to come in contact with skin; an idle roller axially coupled with the front of the body, and rotated to perform an operation of lifting up skin according to an operation of pushing the body while coining in contact with skin; a cam disposed in the body, coupled with a driving motor driving through electrical power supplied from a battery, and eccentrically rotated; and a vibration unit coupled with the outer diameter of the cam, and including a metal ball generating vibration while repeatedly colliding with the inner surface of the contact part according to eccentric rotation of the cam. When the idle roller lifts up skin, the contact part lifts down the skin in the opposite direction of the idle roller through vibration transferred from the vibration unit so as to perform an operation of extending skin between the idler roller and the contact part.

Another patent on Multi-layered massage tool bearing U.S. Pat. No. 1,081,3824B2 is issued to Implus Footcare LLC. The patent is on a multi-layered hand-held massage tool for fascial manipulation includes a weight plate having a first side, a second side opposite from the first side, and an outer edge. The massage tool also includes a first outer layer located on the first side of the weight plate to form a first side of the massage tool, and a second outer layer located on the second side of the weight plate to form a second side of the massage tool. The multi-layered hand-held massage tool has a massage surface, adapted for fascial manipulation, coupling the first side of the massage tool to the second side of the massage tool and covering at least a portion of the outer edge of the weight plate.

Another patent on Massage Tool And Methods Therefor bearing US patent 2,014,0213944A1 is issued to ELC Management LLC. The patent is on a hand-held massage device and methods for applying pressure therapies to various parts of the anatomy using the device are provided. The device is an elongated, rigid, substantially planar member, configured with a series of alternating concave and convex shapes, and a knob-like projecting member at an end thereof.

Another patent on Cylindrical roller massage tool bearing US patent 2,012,0310125A1 is issued to Wendell HallBradley B. PittCherimeer Lynn PittMichele Hall. The patent is on a manually operated cylindrical roller is disclosed which facilitates the even application of pressure to the body tissue of a client during massage therapy, including tendons, ligaments, and muscles (e.g. the gluteus maximus, gluteus medius, sartorius, plantaris, and the like). Variations of the disclosed tool comprise inflatable and non-inflatable components, as well as foam components. The disclosed invention relieves sore and achy muscles, helps blood circulation, relieving muscle cramps, loosening tight and knotted muscles, and relieving pressure from the massage therapist's hands.

A Massage tool bearing US patent 2,015,0272774A1 is issued to Wen-Hwa Lee. The patent is on a massage tool includes a main body; and an active member coupled to the main body. The active member includes an active material capable of being heated for transferring heat to the main body so as to activate or reactivate the massage tool. If necessary, more than one active material may be included in the active member. Heat can be transferred from the active material to the main body by way of conduction of the active material or convection of an active vapor released from the active material. The active vapor, for example, can be steam for providing heat and/or vaporized essential oil for aromatic treatment. The active material, for example, can be heated by an external heating source or an internal heating source.

There are multiple solutions that have been presented in prior art. However, these solutions are limited and restricted to their conventional architecture, installation system and have considerable shortcomings which adversely affect the convenience with which they can be used.

Furthermore, with the advancements in scientific methods, multiple new techniques have been found which have brought numerous benefits to people. One of such inventive approach is Grounding where Grounding or earthing refers to direct skin contact with the surface of the Earth, such as with bare feet or hands, or with various grounding systems. The simplest form involves walking barefoot yin the grass, dirt, or sand. Subjective reports that walking barefoot on the Earth enhances health and provides feelings of well-being can be found in the literature and practices of diverse cultures from around the world. For a variety of reasons, many individuals are reluctant to walk outside barefoot, unless they are on holiday at the beach. Experience and measurements show that sustained contact with the Earth yields sustained benefits. Various grounding systems are available that enable frequent contact with the Earth, such as while sleeping, sitting at a computer, or walking outdoors. These are simple conductive systems in the form of sheets, mats, wrist or ankle bands, adhesive patches that can be used inside the home or office, and footwear. These applications are connected to the Earth via a cord inserted into a grounded wall outlet or attached to a ground rod placed in the soil outside below a window. For the footwear applications, a conductive plug is positioned in the shoe sole at the ball of the foot, under the metatarsals, at the acupuncture point.

After the invention of rubber-soled shoes, a non-conductive barrier was erected between mankind and our greatest source of electrons—the earth. As our direct contact with the earth fades through the routine use of synthetic flooring and shoes, electromagnetic instability threatens our health.

All our cells are made of atoms. Atoms possess unique positive and negative charges that are based on the number of negative electrons or positive protons they carry. Many healthy atoms have a negative charge because they possess more electrons; however, these atoms can have electrons “stolen” from them, leaving them highly reactive and damaging. In this state, they are called free radicals. As damaging free radicals infiltrate cells and tissues, our health declines. The only way to stop this destructive process is by supplying the body with neutralizing antioxidants or a large dose of negative electrons, through grounding

Free radicals are generated through inflammation, infection, cell damage, trauma, stress, and our toxic environments. They force our immune system to respond to these threats. An active immune system produces more free radicals and soon our body is attempting to put out fires, but it has insufficient resources to do so. Additionally, industrialization and our increasingly technological world have thrown us into a labyrinth of electromagnetic fields, which disrupt the electrical balance of our cells. An abundance of free radicals, instable charges, inflammation and immune activation are responsible for some of our most threatening chronic conditions such as cancer, cardiovascular disease, diabetes, chronic pain syndromes, and autoimmunity.

Grounding is a simple, inexpensive means by which most of us can combat these destructive forces. The negative electrons absorbed from the earth quenches the free radicals, supports the immune system, and puts out the fires. Nobel Prize winner Richard Feynman described an umbrella affect created when we “earth.” He claimed that grounding equalized the electronic potential between the body and the earth, so the body becomes an extension of the earth's magnetic field. This potential “cancels, reduces, and pushes away electrical fields from the body.

Therefore, it would be advantageous to design a device which is a simple massager that does not require electricity to operate (manual massager). The device has a conductive component that makes contact with the skin and a conductive wire that connect this conductive component to the grounding port of an electrical outlet. Thus, the proposed assembly uses the method of grounding for massaging and muscle relaxation.

None of the previous inventions and patents, taken either singly or in combination, is seen to describe the instant invention as claimed. Hence, the inventor of the present invention proposes to resolve and surmount existent technical difficulties to eliminate the aforementioned shortcomings of prior art.

### SUMMARY

In light of the disadvantages of the prior art, the following summary is provided to facilitate an understanding of some of the innovative features unique to the present invention and is not intended to be a full description. A full appreciation of the various aspects of the invention can be gained by taking the entire specification, claims, drawings, and abstract as a whole.

The primary desirable object of the present invention is to provide a novel and improved form of a portable massager assembly.

Another object of the present invention is to provide an improved form of a grounded medical device intended to be used as a massage tool.

Another object of this invention is to provide a new and improved assembly which relates to a massage tool, which functions in a more efficient way and achieves better performance than prior art.

It is an object of the invention in one of its aspects to provide a device which can be activated and reactivated so as to be applied in a variety of fields with low cost.

It is also the objective of the invention to provide an assembly which includes a main body; which provides earthing/grounding benefit in addition to a messaging device.

It is also the objective of the invention to provide a smart system which provide ease of use and convenience to everyone and does not requires electricity to operate.

It is a further aspect of the present invention to provide a new and improved assembly which is based on a conductive component that makes contact with the skin and a conductive wire that connect this conductive component to the grounding port of an electrical outlet.

Thus, it is the objective to provide a new and improved assembly of massager device with grounding feature. Other aspects, advantages and novel features of the present invention will become apparent from the detailed description of the invention when considered in conjunction with the accompanying drawings.

This Summary is provided merely for purposes of summarizing some example embodiments, so as to provide a basic understanding of some aspects of the subject matter described herein. Accordingly, it will be appreciated that the above-described features are merely examples and should not be construed to narrow the scope or spirit of the subject matter described herein in any way. Other features, aspects, and advantages of the subject matter described herein will become apparent from the following Detailed Description, Figures, and Claims.

### BRIEF DESCRIPTION OF DRAWINGS

FIG. 1 shows the massagers with the grounding cord incorporated as per preferred embodiments of the invention.

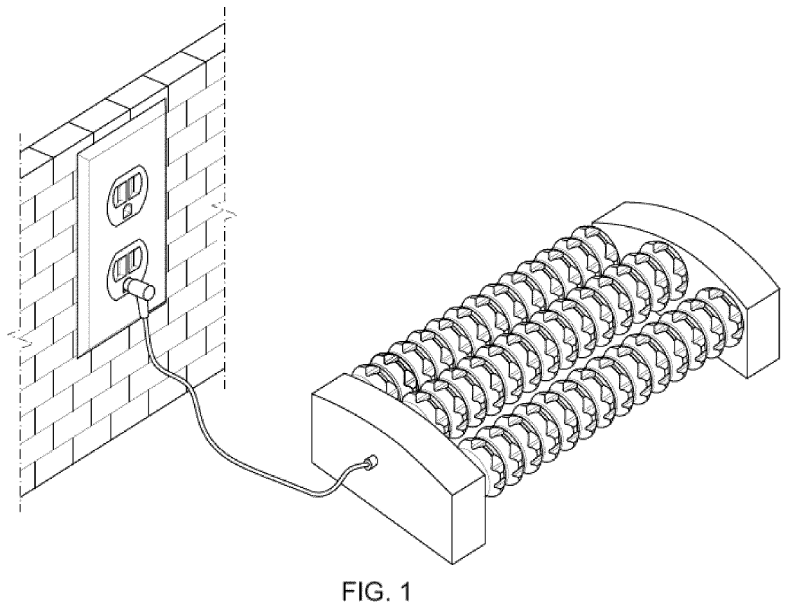

FIG. 2 shows another view of massagers with the grounding cord incorporated as per preferred embodiments of the invention.

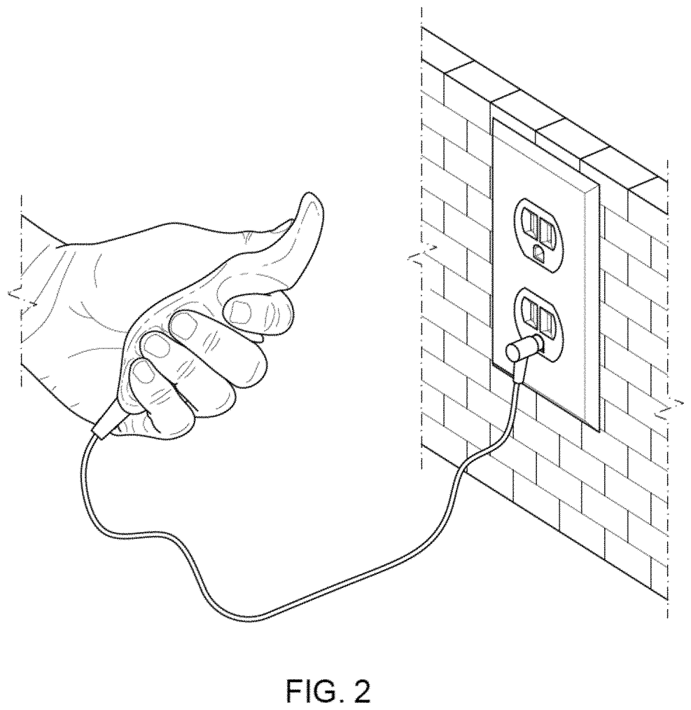

FIG. 3 shows the closeup view of the massager assembly as per preferred embodiments of the invention.

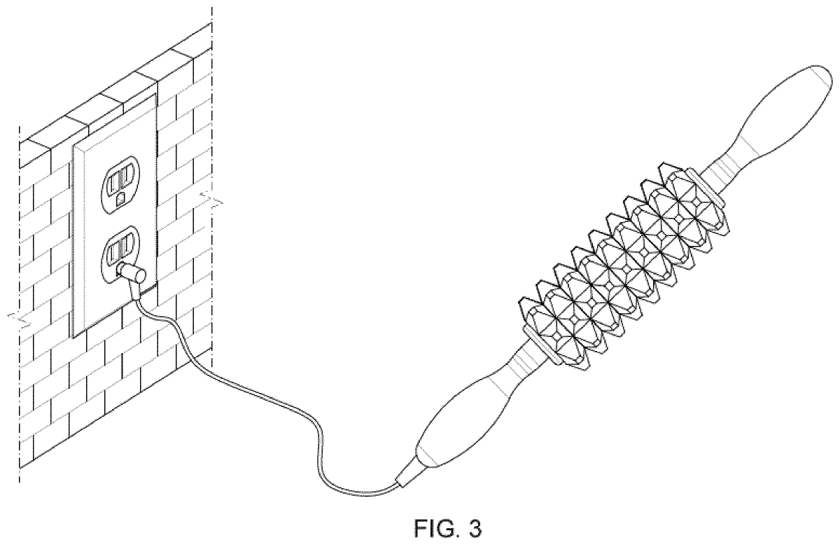

FIG. 4 shows another perspective use of assembly as per preferred embodiments of the invention.

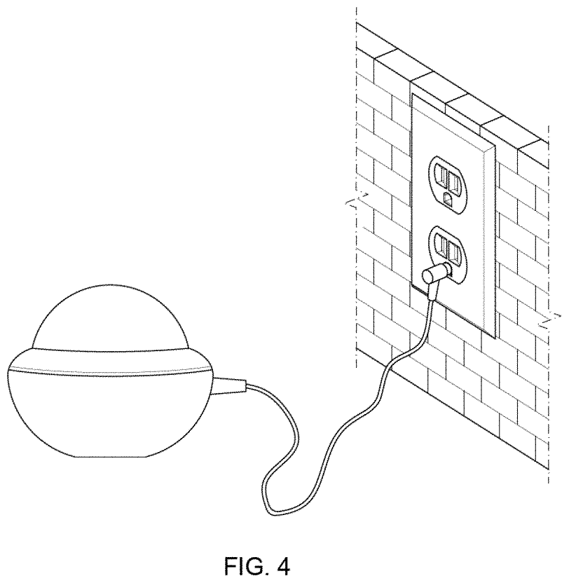

### DETAILED DESCRIPTION

Detailed descriptions of the preferred embodiment are provided herein. It is to be understood, however, that the present invention may be embodied in various forms. Therefore, specific details disclosed herein are not to be interpreted as limiting, but rather as a basis for the claims and as a representative basis for teaching one skilled in the art to employ the present invention in virtually any appropriately detailed system, structure or manner.

The terminology used herein is for the purpose of describing particular embodiments only and is not intended to be limiting of the invention. As used herein, the term “and/or” includes any and all combinations of one or more of the associated listed items. As used herein, the singular forms “a,” “an,” and “the” are intended to include the plural forms as well as the singular forms, unless the context clearly indicates otherwise. It will be further understood that the terms “comprises” and/or “comprising,” when used in this specification, specify the presence of stated features, steps, operations, elements, and/or components, but do not preclude the presence or addition of one or more other features, steps, operations, elements, components, and/or groups thereof.

The present invention is directed to provide an improved grounded medical device intended to be used as a massage tool.

Typically, the human body needs the right amount of blood flow to keep your heart pumping, your legs moving and your brain functioning. Blood circulation, the movement of blood throughout the body, is clearly crucial to your existence, and preventing poor foot circulation should be a high priority.

A person with poor circulation can suffer from a variety of discomforts including pooling of the fluid in the extremities (like the toes), cold hands and feet, fatigue, and achiness created by an accumulation of lactic acid in the muscles. Good circulation brings damaged, tense muscles the oxygen rich blood they need to heal.

Massage facilitates circulation because the pressure created by the massage technique actually moves blood through the congested areas. The release of this same pressure causes new blood to flow in. The squeezing and pulling also flushes lactic acid from the muscles.

The current invention as per its preferred embodiments is a kind of manual massager, comprising the massager's main body and drive accessory, where the described main body comprises of main casing, massage roller-head and be fixedly mounted on driving switch on the main casing, described massage roller-head is to be made off with the many rows of massage contact that is fixed on the axis body that the two ends rolling is installed on the main casing, and flexible massage contact outer end is hemispherical.

While a specific embodiment has been shown and described, many variations are possible. With time, additional features may be employed. The particular shape or configuration of the platform or the interior configuration may be changed to suit the system or equipment with which it is used.

Having described the invention in detail, those skilled in the art will appreciate that modifications may be made to the invention without departing from its spirit. Therefore, it is not intended that the scope of the invention be limited to the specific embodiment illustrated and described. Rather, it is intended that the scope of this invention be determined by the appended claims and their equivalents.

The Abstract of the Disclosure is provided to allow the reader to quickly ascertain the nature of the technical disclosure. It is submitted with the understanding that it will not be used to interpret or limit the scope or meaning of the claims. In addition, in the foregoing Detailed Description, it can be seen that various features are grouped together in various embodiments for the purpose of streamlining the disclosure. This method of disclosure is not to be interpreted as reflecting an intention that the claimed embodiments require more features than are expressly recited in each claim. Rather, as the following claims reflect, inventive subject matter lies in less than all features of a single disclosed embodiment. Thus, the following claims are hereby incorporated into the Detailed Description, with each claim standing on its own as a separately claimed subject matter.

I: An assembly of a manual massager comprising a planar structure with a transverse projection having the shape of mutually parallel teeth attached to a face of said block and two sides on the sides for holding the assembly where the transverse projections provide massaging effect when the massager is moved on the skin of a person and transfers the excess energy from body and thus producing a healing effect at the cellular level.
A massager assembly according to claim I, where the device is any simple massager that does not require electricity to operate.
A massager assembly according to claim I, where the device has a conductive component that makes contact with the skin and a conductive wire that connect this conductive component to the grounding port of an electrical outlet.
A massager assembly according to claim I, where the device helps to reduce the body voltage and making it easier on the cells.
A massager assembly according to claim I, where the device helps to reduce additional anions to specific and targeted areas of the body.
A massager assembly according to claim I, where the device helps to combines the benefits of grounding (earthing) to the benefits of massage.
